(19) United States
(12) Patent Application Publication
(10) Pub. No.: US 2015/0038844 A1
(43) Pub. Date: Feb. 5, 2015

(54) Portable Ultrasound System Comprising Ultrasound Front-End Directly Connected to a Mobile Device

(71) Applicants: Travis Blalock, Charlottesville, VA (US); Michael Fuller, Charlestown, MA (US); Drake Guenther, Charlottesville, VA (US); Karen Morgan, Leesburg, VA (US); Jeff Pompeo, Charlottesville, VA (US)

(72) Inventors: Travis Blalock, Charlottesville, VA (US); Michael Fuller, Charlestown, MA (US); Drake Guenther, Charlottesville, VA (US); Karen Morgan, Leesburg, VA (US); Jeff Pompeo, Charlottesville, VA (US)

(21) Appl. No.: 13/957,155

(22) Filed: Aug. 1, 2013

(51) Int. Cl. A61B 8/00; A61B 8/14; A61B 8/08

(52) U.S. Cl. CPC A61B 8/4427; A61B 8/463; A61B 8/5207; A61B 8/5223; A61B 8/4477; A61B 8/145
USPC 600/440; 600/459; 600/447; 600/449

(57) ABSTRACT

A portable ultrasound imaging system includes a mobile computing device; a detachable front end component configured for attachment to and communication with the mobile computing device, and configured to transmit and receive ultrasound signals; and programming, when installed on the mobile computing device, being executable by the mobile computing device to cause the mobile computing device to send signals to the front end component causing the front end component to transmit the ultrasound signals, and to receive signals from the front end component resulting from the front end component receiving the receive ultrasound signals, and process the receive signal and display an ultrasound image resulting from the processing. The front end component is configured to be directly joined with the mobile computing device and directly connected, without the use of an external wire or cable.

### FIELD OF THE INVENTION

The present invention relates to the field of ultrasound imaging and is particularly well suited to the field of medical imaging. More specifically, the present invention relates to devices, methods and computer readable media for portable, ultrasound imaging.

### BACKGROUND OF THE INVENTION

Medical imaging is a field in which imaging systems are predominately very high cost and complex enough to require operation and interpretation by experienced and highly trained medical staff. Medical ultrasound is generally considered a low cost imaging modality within the medical imaging field but utilizes imaging systems costing as much as $250K. These high-tech, high-cost systems are useful for diagnostic ultrasound exams, however the cost and training requirements limit their use in many routine exams for which ultrasound can be clinical useful.

Over the past two decades a number of companies have attempted to develop low-cost, easy-to-use ultrasound systems for use in non-radiology settings for routine use. An example is Sonosite, which was the first to sell hand-carried ultrasound systems at lower costs. While far less expensive than high-end systems and much more portable, these systems are still fairly sophisticated and require a well-trained operator who can mentally map the image plane on the screen to the anatomy being imaged and adjust a large set of system parameters to optimize image quality.

Other companies have followed the success of the portable ultrasound systems and continue to make smaller and less costly systems. All of these systems, though, are still fundamentally miniaturized versions of their fully featured predecessors and the image formation and the separation between the ultrasound transducer and the system display require more training to be able to interpret the images. They system described here takes the approach of bringing the image closer to the anatomy of interest, by displaying the image in close proximity to the anatomy and in the same orientation as the anatomy.

Typically, currently available portable ultrasound systems have a display of some sort that is separated by a long cable from the portion of the system in contact with the patient that transmits and receives the ultrasound signals (the ultrasound transducer). These cables can be cumbersome for the operator and also add to the expense of the system for high channel count transducers, while at the same time making them relatively less portable. Furthermore, the separation between the transducer and the system display requires the operator to turn his/her attention away from the patient to view the display. This is particularly challenging during ultrasound-guided procedures.

The B-Mode image format is what most conventional ultrasound systems use and it is a representation of a slice through the body perpendicular to the transducer face (or the skin surface). This image format is less intuitive because as the transducer is moved the operator has to mentally reconstruct the image slices to understand the volume being interrogated for the anatomy below.

Several handheld ultrasound devices have been described that can be characterized as standalone systems that do not utilize commercially available mobile devices. U.S. Patent Application Publication Nos. 2009/0198132 A1 and 2012/0232380 A1 to Pelissier et al describe a hand-held ultrasound imaging device built as a dedicated, integrated custom unit. The system described in U.S. Patent Application No. 2009/0198132 A1 has an integrated transducer, whereas the system described in U.S. Patent Application No. 2012/0232380 A1 has a detachable transducer. Both of these systems are fully custom, including the display, user interface and processing components. U.S. Pat. No. 6,139,496 to Chen and Atlas describes a custom ultrasound imaging system where the insonifying transducer elements and display units are integrated into a probe assembly that is connected via a cable to a control and data processing unit. One ultrasound system described in U.S. Patent No. 2008/0208061 A1 specifically mentions a pocket-sized ultrasound imaging system utilizing a custom hand-carried device with an I/O port to attach a cabled transducer probe. U.S. Pat. No. 7,699,776 B2 to Walker et al describes a standalone handheld ultrasound system that performs C-mode imaging and collects 3D image data using a 2D transducer array that is integrated into the ultrasound system without a separate cable connection.

Several other handheld ultrasound devices have been described that are not standalone systems, but instead utilize commercially available mobile devices to handle functions such as display, user interface, and processing. U.S. Patent Application Publication No. 2007/0239019 A1 to Richard et al describes an ultrasonic imaging probe consisting of an ultrasound transducer, front-end receive circuitry, logarithmic compressor, envelope detector and interface circuitry that communicates with, receives power from, and connects to a host computer via a passive interface cable. U.S. Patent Application No. 2011/0054296 A1 to McCarthy et al describes using a commercially available mobile device as a remote display that is tethered by way of a cable to a separate display and processing unit and ultrasound probe. U.S. Patent Application No. 2003/0097071 A1 to Halmann et al describes a handheld ultrasound system consisting of a beamforming module with detachable transducer head that interfaces with a personal digital assistant (PDA) device. U.S. Patent Application Publication No. 2013/0003502 A1 to Prakash et al describes an ultrasound Doppler transceiver that may be integrated with a mobile computing device. This device is limited to making Doppler measurements, such as finding the velocity of a target object or monitoring an in utero baby's heart rate; it does not form 2D or 3D ultrasound images.

Other inventions describe cases or housings for ultrasound systems that utilize commercially available mobile devices. U.S. Design Pat. No. D657,361 S to Goodwin et al describes an ornamental design for a housing surrounding a mobile device. Although not specifically covered by the design patent, the drawings show a transducer attached to the housing via a cable.

### SUMMARY OF THE INVENTION

In one aspect of the present invention, a portable ultrasound imaging system includes: a mobile computing device; a detachable front end component configured for attachment to and communication with the mobile computing device, and configured to transmit and receive ultrasound signals; and programming, when installed on said mobile computing device, being executable by the mobile computing device to cause the mobile computing device to send signals to the front end component causing the front end component to transmit the ultrasound signals, and to receive signals from the front end component resulting from the front end component receiving the receive ultrasound signals, and process the receive signal and display an ultrasound image resulting from the processing; wherein the front end component is configured to be directly joined with the mobile computing device and directly connected, without the use of an external wire or cable.

In at least one embodiment, at least a portion of the front end component is movably mounted to the mobile computing device to allow relative rotation about at least one axis of rotation relative to the mobile computing device.

In at least one embodiment, the mobile computing device is a device selected from the group consisting of: a smartphone, a tablet computing device, and a personal digital assistant (PDA).

In at least one embodiment, the mobile computing device comprises a smartphone.

In at least one embodiment, the mobile computing device comprises a tablet computing device.

In at least one embodiment, at least a portion of the front end component is movably mounted to the mobile computing device to allow relative rotation about at least two axes of rotation relative to the mobile computing device.

In at least one embodiment, at least a portion of the front end component is movably mounted to the mobile computing device to allow relative rotation about three axes of rotation relative to the mobile computing device.

In at least one embodiment, the ultrasound image is displayed in real-time.

In at least one embodiment, the front end component further comprises a barrier element that shields the mobile computing device from contact with a patient when the front end component is applied to a patient.

In at least one embodiment, the barrier element forms a seal with the mobile computing device to provide a sterile barrier.

In at least one embodiment, the system further includes a locking element configured to fix the front end component relative to the mobile computing device to maintain a desired orientation of the front end component relative to the mobile computing device.

In at least one embodiment, the programming is configured so that, when a position of front end component relative to the mobile computing device is changed, the processor executes the programming to change a display mode of an image being displayed.

In at least one embodiment, the front end component comprises a two-dimensional ultrasound transducer.

In at least one embodiment, execution of the programming by the processor processes the receive signals to form an image similar to an image that would otherwise be formed by processing signals received from a front end component employing a one-dimensional transducer.

In at least one embodiment, the front end component comprises a one-dimensional ultrasound transducer.

In at least one embodiment, the front end component comprises multiple distinct transducer arrays which are capable of acquiring two separate sets of ultrasound data, each the distinct transducer array being configured to transmit and receive distinct ultrasound signals.

In at least one embodiment, a first of the two distinct transducer arrays operates at a first center frequency, and a second of the two distinct transducer arrays operates at a second center frequency, wherein the second center frequency is different from the first center frequency.

In at least one embodiment, a first of the two distinct transducer arrays is a one-dimensional transducer array, and a second of the two distinct transducer arrays is a two-dimensional transducer array.

In at least one embodiment, the two distinct transducer arrays are oriented in different directions on the front end component.

In another aspect of the present invention, a front end component is provided that is configured for communication with a mobile computing device to function as a portable ultrasound imaging system, the front end component including: a main body configured and dimensioned to fit over the mobile computing device; a mating connector configured and dimensioned to directly mate with a connector on the mobile computing device for direct connection of the front end component to the mobile computing device without any need for a connection wire or cable; and a transducer array movably mounted relative to the main body, to allow relative rotation of the transducer array about at least one axis of rotation relative to the main body; wherein the main body is configured to form a seal with the mobile computing device.

In at least one embodiment, the transducer array is configured for a predetermined footprint and element pitch; and wherein the front end comprises at least one application specific integrated circuit (ASIC) configured to enable a front end dimensional footprint and front end channel pitch, wherein the front end dimensional footprint and the front end channel pitch match the footprint and element pitch, respectively.

In another aspect of the present invention, a method of operating a portable ultrasound imaging system includes: directly connecting a front end device to a mobile computing device, without the use of an extension cable or wire, the front end component including a transducer array, configured for communication with the mobile computing device, and configured to transmit and receive ultrasound signals; positioning the transducer array over a location of a target to be imaged; selecting custom software installed on the mobile computing device to be used in performance of imaging; selecting imaging settings on the custom software; activating the custom software; propagating acoustic signals toward the target to be imaged; receiving acoustic signals reflected off of the target to be imaged; converting the acoustic signals received to digitized electrical signals; processing the digitized electrical signals; and displaying an image of the target on a display of the mobile computing device.

In at least one embodiment, the transducer, in a first angular orientation relative to the display, causes the custom software to display in a first imaging mode.

In at least one embodiment, the method further includes changing the transducer to a second angular orientation relative to the display, wherein the second angular orientation causes the custom software to display the image in a second imaging mode different from the first imaging mode.

In another aspect of the present invention, a non-transient computer readable medium including one or more sequences of instructions for performing ultrasound imaging system on a portable ultrasound imaging system, wherein execution of the one or more sequences of instructions by one or more processors of the portable ultrasound imaging system causes the portable ultrasound imaging system to perform a process including: setting imaging settings for an imaging process to be performed on a mobile computing device loaded with the one or more sequences of instructions, and directly connected to a front end device including a transducer array; sending commands from the mobile computing device to a transmit and receive control module in the front end device; controlling ultrasound circuitry to transmit ultrasound signals in accordance with the imaging settings to the transducer array; propagating acoustic signals into a target to be imaged; receiving acoustic signals having been reflected off the target to be imaged; converting the acoustic signals received to electrical signals; processing the electrical signals; and displaying an image of the target on a display of the mobile computing device.

In at least one embodiment, the non-transient computer readable medium further includes instructions which, when executed by the portable ultrasound imaging system, cause the system to: display the image in a first imaging mode when the transducer is in a first angular orientation relative to the display; and, upon changing orientation of the transducer array relative to the display to a second angular orientation different from the first angular orientation, displaying the image in a second imaging mode different from the first imaging mode.

These and other advantages and features of the invention will become apparent to those persons skilled in the art upon reading the details of the systems, components, methods and computer readable media as more fully described below.

### BRIEF DESCRIPTION OF THE DRAWINGS

FIG. 1A is a schematic representation of a system according to an embodiment of the present invention

FIG. 1B schematically illustrates software included within a system according to an embodiment of the present invention.

FIG. 2 is an exploded illustration of a system according to an embodiment of the present invention.

FIG. 3A is an unassembled view of a system according to an embodiment of the present invention.

FIG. 3B is an assembled view of the system of FIG. 3A.

FIG. 4 is a flow chart illustrating events that may occur during operation of a system according to an embodiment of the present invention.

FIGS. 5A-5C are three different perspective views of a system illustrating angular adjustability of the transducer relative to the mobile computing device and front and back walls of the front end device, according to an embodiment of the present invention.

FIGS. 5D-5E show a system wherein the transducer array is oriented at an angle of zero degrees relative to the mobile computing device 500, and a representation of a C-Mode image obtained thereby, according to an embodiment of the present invention.

FIGS. 5F-5G show a system wherein the transducer array is oriented at an angle of zero degrees relative to the mobile computing device 500, and a representation of a C-Mode image obtained thereby, as a different depth relative to the depth of the image taken in FIGS. 5D-5E, according to an embodiment of the present invention.

FIGS. 5H-5I show a system wherein the transducer array is oriented at an angle of zero degrees relative to the mobile computing device 500, and a representation of a Volume-Mode image obtained thereby, according to an embodiment of the present invention.

FIGS. 5J and 5K show the transducer array oriented at an angle of about ninety degrees relative to the mobile computing device of the system according to an embodiment of the present invention.

FIGS. 6A-6F illustrate respective front and side views of a system according to another embodiment of the present invention, in three different exemplary, but non-limiting use orientations.

FIGS. 6G-6H illustrate an example of a locking element according to an embodiment of the present invention.

FIGS. 7A-7F illustrate a system in which transducer assembly is mounted on the end of the mobile computing device and front end device according to another embodiment of the present invention,

FIGS. 8A-8B illustrate a system wherein the mobile computing device comprises a tablet computer, according to an embodiment of the present invention.

FIG. 9 illustrates a system wherein the front end device is provided with two transducer arrays according to an embodiment of the present invention.

### DETAILED DESCRIPTION OF THE INVENTION

Before the present systems, programming, methods and computer readable media are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a transducer” includes a plurality of such transducers and reference to “the battery” includes reference to one or more batteries and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

The term “B-mode image” as used herein, refers to an image resultant from B-mode ultrasonography, in which a position of a spot on the image display corresponds to an elapsed time (from time of sending an ultrasound pulse/wave until time of receipt of the echoed, ultrasound pulse/wave, and thus to the position of the echogenic surface off which the ultrasound pulse/wave was reflected) and the brightness of the spot corresponds to the strength of the echo and is in a plane roughly perpendicular to the surface.

The term “C-mode image”, as used herein, refers to a two-dimensional image formed in a plane approximately parallel to the surface of the transducer at constant distance from the ultrasonic transducer or depth.

The term “azimuth” generally refers to the axis in the direction along the long side of the transducer array.

The term “elevation” generally refers to the axis in the direction along the short side of the transducer array.

The term “footprint” as used herein, refers to the surface space occupied by a structure (e.g. the area of an element in the transducer array, or the area occupied by the entire transducer array.)

The term “pitch”, as used herein, refers to the center to center distance of two adjacent structures (e.g. distance between centers of two adjacent elements in a transducer array or distance between centers of two adjacent front-end receive channels in the custom ASIC.).

The phrase “mobile computing device” refers to a mobile computing device that is not specifically designed, nor is it produced in a configuration for performing ultrasound scans. Rather it is a mobile device manufactured for general computing, for performing functions the same as or similar to a desktop computer such as a PC or Apple desktop computer. Additional functions may include use as a telephone, for example. Examples of “mobile computing devices” are those having been manufactured for use by the general population including, but are not limited to: tablet computers, such as the IPAD (Apple Computer, Cupertino, Calif.), Kindle (Amazon), or other tablets, such as those produced and readily available for general use by the public, such as by Samsung, Microsoft, etc.; smartphones, such as the iPHONE (Apple Computer, Cupertino, Calif.) smartphones operating on the ANDROID operating system (Google, Mountain View, Calif.), or other smartphone; personal digital assistant (PDA) device (e.g., iPOD Touch (Apple Computer), or other PDA), and the like.

“Real time”, as used herein, refers to a system that can acquire and process data fast enough to enable control of the source of the data. So for example, on our device real-time imaging means that the user can see images from a particular transducer position and orientation quickly enough that the user can use that information immediately to reposition the transducer.

### DESCRIPTION

The present invention provides a portable ultrasound system that is compact and, when assemble is fully integrated with no cables. The system is simple to operate and the user does not need to direct his/her attention away from the patient in order to interpret the images provided by the system, while operating the system. The user interface is simple and intuitive, easy to operate.

The present invention system utilizes a mobile device, which is readily publicly available and mass produced at low costs. A front end component according to an embodiment of the present invention, configured for communication with the mobile computing device, and configured to transmit and receive ultrasound signals is directly connected to the mobile computing device, without the use of an extension wire or cable. Programming, when installed on the mobile computing device, is executable by the mobile computing device to cause the mobile computing device to send signals to the front end component causing the front end component to transmit ultrasound signals, and to receive signals from the front end component resulting from the front end component receiving receive ultrasound signals, and process the receive signals and display an ultrasound image resulting from the processing on the display of the mobile computing device.

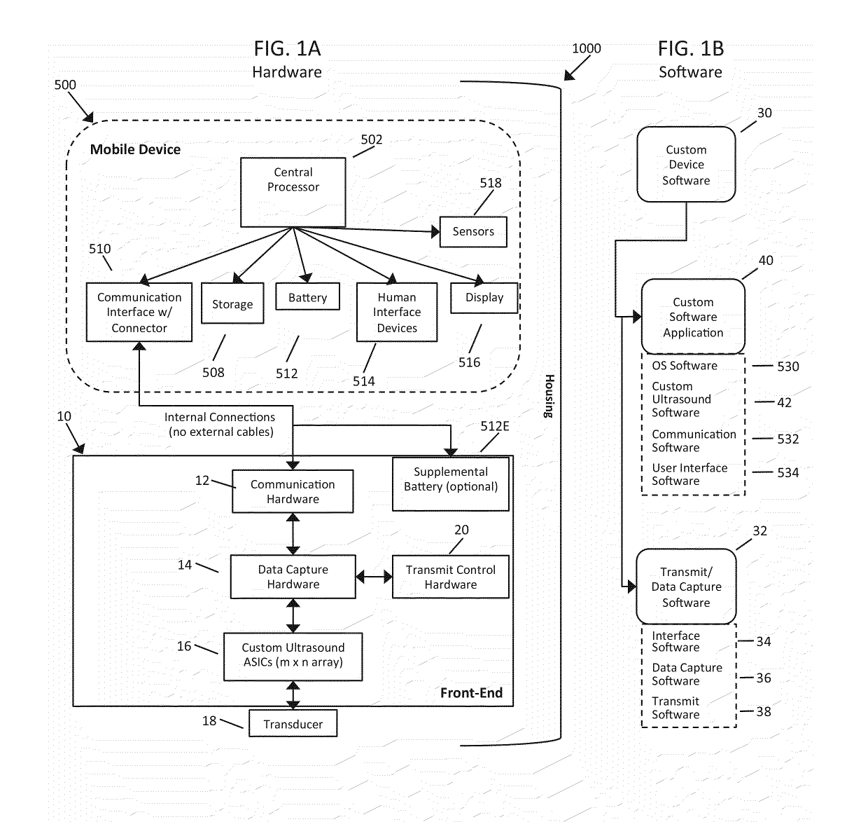

FIG. 1A is a schematic representation of a system 1000 according to an embodiment of the present invention, indicating hardware and software components that are included in the system, although the system is not necessarily limited to the hardware and software shown. FIG. 1B schematically illustrates software included within the system 1000, according to an embodiment of the present invention. The mobile computing device 500 includes one or more processors 502 (also referred to as central processing units, or CPUs) that are coupled to storage devices 508 (which may include primary storage, such as a random access memory, or RAM, primary storage such as read only memory, or ROM, and mass storage). As is well known in the art, ROM primary storage acts to transfer data and instructions uni-directionally to the CPU 502 and RAM primary storage is used typically to transfer data and instructions in a bi-directional manner. The storage devices 508 can also include a mass storage device that is also coupled bi-directionally to CPU 502 and provides additional data storage capacity. The mass storage device in 508 may be used to store programs (including but not limited to custom software application 40, data from which can also be transferred or loaded to the primary storage in 508), data and the like. Alternatively, primary storage can be combined with mass storage and provided as solid state memory. It will be appreciated that the information retained within the mass storage device in 508, may, in appropriate cases, be incorporated in standard fashion as part of primary storage in 508 as virtual memory.

CPU 502 is also coupled to an interface (communication interface device) 510 that includes a connector for physically and directly connecting the custom front end device 10 thereto. For example, pin to socket connections can be made in a direct connection, without the use of any extension cable interconnecting the mobile computing device 500 and front end device 10. Alternatively, inductive or capacitive interfaces could be employed, which may not require a direct pin-to-socket connection. Mobile computing device 500 further includes as least one human interface device 514, which is typically a touch screen or integrated keyboard. Additionally or alternatively, one or more devices such as video monitors, track balls, mice, external keyboards, microphones, touch-sensitive displays, transducer card readers, magnetic or paper tape readers, tablets, styluses, voice or handwriting recognizers, or other well-known input devices such as, of course, other computers, may be employed, but are optional and not required.

The mobile computing device 500 typically include sensors 518, such as accelerometers, gyroscopic/positional sensors, compasses, thermometers, light sensors, cameras, GPS, proximity sensors, RFID readers, and other well-known sensing components. These sensors can be used in a human interface context (mentioned in the previous paragraphs) or non-human-interface context to augment the ultrasound imaging process, particularly for a system 1000 comprised of a physically integrated front end device 10 and mobile computing device 500. Examples include changing image plane orientation or acquisition based on the sensing of tilt, translation, or rotation of the imaging system 1000, detecting and removing (or enhancing) motion artifacts, and using cameras for optical tracking.

A display 516 is preferably included in device 500 and is used by the present invention to display images resultant from ultrasound procedures as described below. Additionally and optionally, data resulting from such processing and used to display images can be output to another device, such as an external display, printer or the like.

A battery 512 is typically provided in the mobile computing device and connected with the other components so as to power the operation of the CPU 502 and other components of the device 500. Optionally, a supplemental battery 512E may be provided as a part of the front end module 10 to supplement the power supply provided by the mobile device 500.

Further additionally and optionally, data resulting from such processing and used to display images can be transmitted to another computing device or external output device, via internet or wirelessly, preferably wirelessly. For example, device 500 optionally may be coupled via CPU 502 and known interface devices (wired or wireless) to a computer or telecommunications network. With such a network connection, it is contemplated that the CPU 502 might receive information from the network, or might output information to the network in the course of performing the methods described herein.

The front end device 10 is configured to readily and directly connect to the mobile computing device 500 without the need for any additional connection hardware or cable. Front end device 10 includes communication hardware 12 that includes a connector configured and dimensioned to mate with the connector of the communication interface 510 to directly connect and mount front end device 10 to mobile computing device 500, so that no external connection wire or cable is required. The communication hardware 12 is dictated in several ways by the communication interface 510. From a physical standpoint, the size, shape, and pin-out (function, size, pitch, and position of pins or other physical electrical power or signal terminations) of the communication hardware 12 connector constrains the means by which the communication hardware 12 can mechanically and electrically mate or interface with the mobile device 500. From a functional standpoint, the communication protocol employed by the communication interface 510 determines the hardware specifications necessary to implement this protocol (e.g. signal bandwidth, voltage/current requirements, analog vs. digital signaling, single-ended vs. differential, etc.) as well as the interface software 34 requirements. For example, many mobile devices to date use the Universal Serial Bus (USB) 2.0 standard, which specifies the cables, connectors, power supply parameters, and communication protocol. A mobile device communication interface 510 may follow the USB 2.0 standard in full, or it might follow only a portion of the standard, choosing to modify the mechanical, electrical, or communication protocol elements according to proprietary specifications. Alternatively, the communication interface 510 might be based on alternative industry standard or proprietary standard. Whatever communication interface 510 exists on the mobile device 500, the custom front-end 10 must cater the design of its communication hardware 12 such that it is mechanically, electrically, and functionally compatible with that of the communication interface 510. All or only a portion of the available pins, mechanical features, or functional characteristics of the communication interface 510 may or may not be useful to the purposes of the custom front-end 10.

Data capture hardware 14 is custom hardware which includes a programmable device which brokers configuration and data commands and requests originating from the communication hardware (12), passing them on to the custom ASICS (16). The data capture hardware can take several different forms, but in general it must be capable of deterministic synchronous I/O timing not subject to interrupts or non-deterministic delays common to most microprocessors. Examples of suitable data capture controllers include field programmable gate arrays (FPGAs), complex programmable logic devices (CPLDs), or microcontrollers, which contain synchronous memory buffers.

A custom ultrasound circuit (such as an application specific integrated circuit (ASIC), for example) 16 consists of front-end receive channels whose function is minimally to amplify, sample and digitize the electrical signal originating from the transducer elements making up the transducer array 18 (created by electromechanical conversion of the received acoustic echo pulse) to which it is connected. A receive channel may only be connected to a single, unique transducer element or multiple transducer elements might share the same receive channel through multiplexing. The receive channel circuit components commonly consist of a low-noise preamplifier, a variable gain amplifier (VGA), a low-pass or band-pass filter, a sample-and-hold (S/H) unit, an analog-to-digital converter (ADC), and digital memory. High voltage switches or protection circuitry is often required to protect the receive circuitry (often implemented in a low-voltage IC process) from the high-voltage transmit pulse. The ADC and digital memory components may be implemented in every channel, shared by multiple channels, or implemented off-chip. An example of such a custom ultrasound ASIC is described in U.S. Pat. No. 8,057,392 to Blalock et al entitled, “Efficient architecture for 3D and planar ultrasonic imaging—synthetic axial acquisition and method thereof”, which is hereby incorporated herein, in its entirety, by reference thereto. The custom ultrasound ASICs 16 must be custom designed to enable the dimensional footprint and channel pitch to match the footprint and element pitch of the transducer array 18, which is critical to enabling the connection of these components (16 and 18) using advanced integrated circuit (IC) packaging techniques, which in turn eliminates the need for the cable-based connections typical of conventional ultrasound systems. The custom circuitry of the ultrasound ASICs 16 must also meet several specifications unique to the custom front-end 10, including, but not limited to, center frequency, frame-rate, samples acquired per frame, gain adjustment range, data readout bandwidth, and power budget.

A transducer or plurality of transducers arranged in an array 18 is provided to communicate with the custom ultrasound circuit 16, to transmit ultrasound energy from the transducer array 18 in accordance with electrical signal input received from the circuit 16, and to send electrical signals converted from reflected ultrasound energy received by the transducer array 18. Throughout we refer to the transducer or plurality of transducers arranged in an array as the transducer array 18. Transducer array 18 may be externally mounted on the front end device 10. Alternatively, transducer array may be incorporated within the body of the front end device 10. The transducer array footprint and element pitch must match or approximate the footprint and receive channel pitch of the custom ultrasound ASICs 16 in order to enable direct electrical and mechanical integration of these two components 16 and 18 in such a way that the cable or cables commonly used in most ultrasound systems to connect transducer elements to receive channels are eliminated. The direct electrical and mechanical connection can be implemented using a variety of IC packaging methods and technologies that are well known to those skilled in the art, including wire-bond, flip-chip, and wafer-level packaging (WLP) methods. Since the ultrasound ASICs 16 are custom, this means the transducer array 18 will be custom, unless the ultrasound ASICs 16 are designed to be integrated with an existing proprietary or commercially available transducer array 18. The transducer array 18 can be a separate component that is later integrated with the custom ultrasound ASICs 16, or it can be built directly on the custom ultrasound ASICs 16, on a substrate containing the custom ultrasound ASICs 16, or on its own substrate that is itself later integrated with the custom ultrasound ASICs 16 or custom ultrasound ASICs substrate.

Transmit control hardware 20 is connected to and communicates with the data capture hardware 14 (or alternatively it could connect and communicate with the communication hardware 12 or another hardware component not shown) with the function of generating the transmit signal and driving the transmit signal onto the transducer array 18. Those skilled in the art will recognize the transmit signal may be a single signal that drives a single transducer element or is shared by multiple transducer elements, or it may consist of multiple transmit signals each driving one or more transducer elements. For the latter case of multiple transducer signals, each signal typically differs in terms of phase (for the purpose of focusing on transmit), but can also differ in amplitude, frequency, bandwidth, and other characteristics. The timing of the transmit signal must be carefully controlled not only in terms of transducer resonance and bandwidth, but also in terms of the front end receive circuit timing and any relative phase delays of multiple transmit signals for the case of focusing on transmit. This precise control over timing typically necessitates the use of a microcontroller or FPGA with sufficient clock speed and deterministic I/O timing. The transmit signal is typically, but not always, a high-voltage signal often exceeding 5 Vpp (typically >100 V), in which case discrete high-voltage switches (e.g. field-effect transistors (FETs), bipolar junction transistors (BJTs)) or integrated high-voltage driver circuits are required to drive the transducer element or elements and logic level translator components are necessary to enable interfacing to lower-voltage (typically <5 V) controller circuitry such as the data capture hardware 14.

Custom device software 30 is implemented in both the front end device 10 and in the mobile computing device 500 to control and coordinate ultrasound processing in accordance with embodiments of the present invention. In the front end device, data capture programming/software is provided for control of transmission of ultrasound energy from the transducer array 18 (via the control circuit 16), as well as for control of data capture from the electrical signals received from the transducer array 18 (via ultrasound control circuit 16). The custom software residing on the Front End hardware 10 is referred to general as the Transmit/Data Capture Software 32. This includes Interface Software 34, Data Capture Software 36 and Transmit Software 38 as generally described in the following paragraphs.]

The data capture software 36 generally resides on the data capture hardware 14 and serves to properly configure the front end ASICs for accurately sampling the returning acoustic data. The custom software on the mobile device 40 passes ultrasound receive parameters (e.g. sampling time, number of samples, channel gain, etc.) and instructions to the data capture software 36 via the interface software 34 and these instructions are translated into the specific signaling and low-level interactions with the front end circuits required to implement the function requested. Upon completion of the requested receive operation, digitized acoustic data is then read out from the front end ICs via the data capture software 36 and hardware 14, and the data undergoes further processing (e.g. error decoding, sorting, signal conditioning, etc.) prior to being passed back to the mobile device through the communication hardware. Once the data has been successfully transferred back to the mobile device, the data capture software prepares the front-end chips for the next receive operation.

Interface software (programming) 34 is provided in front end device 10 to provide a simple interface for the higher level application software on the mobile device to communicate with the transmit/receive hardware. The interface software resides on the communication hardware 12 and the protocol will depend on the protocol used by the mobile device (for example USB). This interface software allows for the transfer of commands from the mobile device software to the front end PCB to properly configure the front end hardware for the transmission of acoustic energy and the reception and digitization of the returning ultrasound echoes. The interface software also allows for the transfer of ultrasound echo data, acquired on the front end, to the mobile device CPU 502.

Transmit Software 38 in conjunction with the Transmit Control Hardware 20 is provided on the front end device 10 to generate the transmit driving signal, which electrically drives the ultrasound array emitting an acoustic pulse. The Transmit Software 38 properly configures the Transmit Control Hardware 20 to transmit an acoustic pulse with desired characteristics such as amplitude, frequency, bandwidth and timing across the array elements in the case of transmit focusing.

The mobile computing device is programmed with a custom software application 40 that is executable by CPU 502 via the operating system software 530 that the device 500 is provided with as generally available to the public. Custom software application 40 is executable and interfaces with the communication software 532 and user interface software 534 of the device 500. The software application includes the communication software 532, custom ultrasound software 42, user interface software 534, and OS software 530 needed to interface with the mobile device. The custom software application controls the operations of the front end hardware 10, receives and interprets commands from the human interface devices 514 using the user interface software 534, and processes and displays images resulting from ultrasound procedures and other aspects of ultrasound procedures described herein, using custom ultrasound software 42 provided in custom software application 40. The custom software application 40 may be downloaded to the mobile computing device 500 in any manner currently available for what is commonly referred to as “downloading apps”, or may be programmed into the device 500 by numerous other software uploading techniques that would be readily apparent to one of ordinary skill in the art.

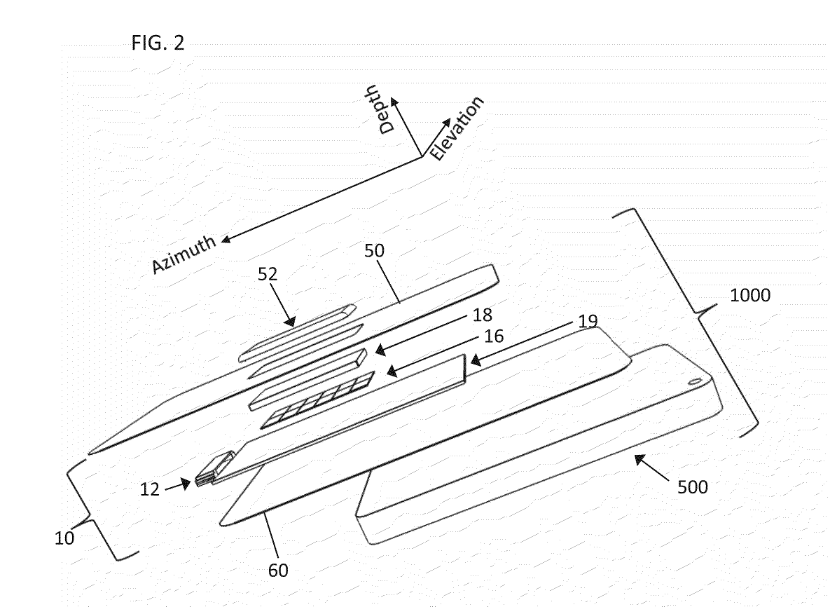

FIG. 2 is an exploded illustration of a system 1000 according to an embodiment of the present invention. In this embodiment, the ultrasound transducer assembly 18 is provided within the main body of the front end device 10 and is sandwiched between the outer wall 50 of device 10 and the ultrasound control circuit 16, as shown. Transducer assembly 18 is mounted internally and parallel to an acoustic window 52 provided in outer wall 50 of front end device 10. Acoustic window 52 is acoustically transparent to the ultrasonic frequencies employed in the ultrasonic procedures described herein, so as to allow the ultrasonic energy to freely, bi-directionally pass therethrough without any substantial interference to or distortion of the ultrasonic energy passing therethrough. In the configuration illustrated in FIG. 2, transducer assembly 18 would also be oriented substantially parallel to the back (and front) surface(s) of the mobile computing device 500. For the transducer array 18 orientation shown, the azimuthal and elevational directions are represented on axes shown in FIG. 2, and the depth direction is illustrated on the third axis of the three-dimensional, orthogonal axis system shown.

The custom ultrasound control circuitry 16 is also contained within the front end device 10 and, in this embodiment is located between the transducer array 18 and back wall 60 of the device 10, on printed circuit board 19. The communication hardware 12 includes a connector configured and dimensioned to mate with and directly connect to the connector 510 provided to the mobile computing device 500. Connector/communication hardware 12 thus electrically connects with the mobile computing device 500 and further, is electrically connected to the ultrasound control circuitry 16 as shown in FIG. 2.

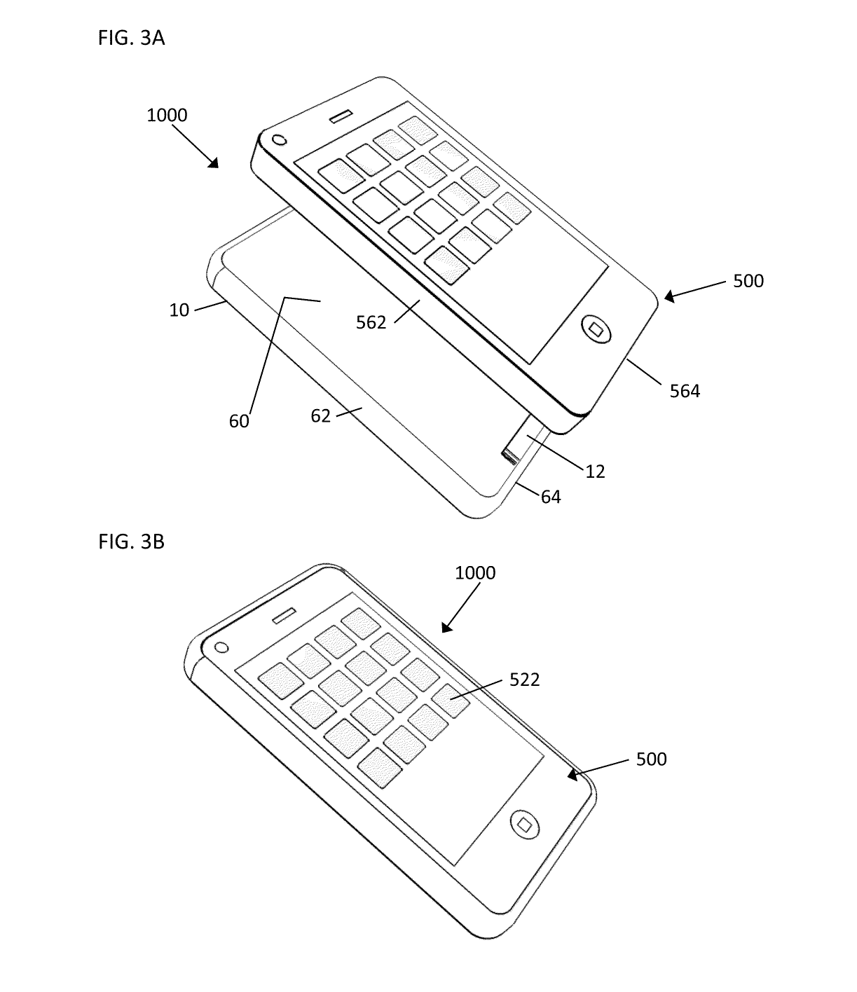

The main body of the front end device 10 may further be provided with side and end walls 62, 64, respectively (e.g., see FIG. 3A), that extend upwardly beyond the back wall 60 so as to cover the side and end walls 562, 564, respectively, of the mobile computing device 500 when the system 1000 is assembled, as shown comparatively in the unassembled view of FIG. 3A and the assembled view of FIG. 3B. At least the side and end walls are somewhat flexible and resilient so that they can be deformed to allow the connector of the communication hardware 12 to be plugged into the connector of the communication interface 510 to assemble the front end device 10 to the mobile computing device 500 as shown in FIG. 3B, to form the system 1000. After plugging in the connector of the communication hardware 12, the side and end walls 62, 64 resiliently return to their preconfigured orientations which are substantially perpendicular to the wall 60, and conform to the side and end walls of the device 500, thereby forming a seal with the mobile computing device 500 to provide a sterile bather therewith and preventing contamination to the side and end walls, and back surface of the mobile computing device 500. Alternatively, the mobile computing device 500 may be completely protected by a barrier that not only covers the side and end walls, but also covers the display face, such as with a clear layer that allows visualization of the display and operation of the touch screen or other input devices present. Still further, the barrier would completely envelop and seal the mobile computing device in an alternative embodiment to prevent it from contamination.

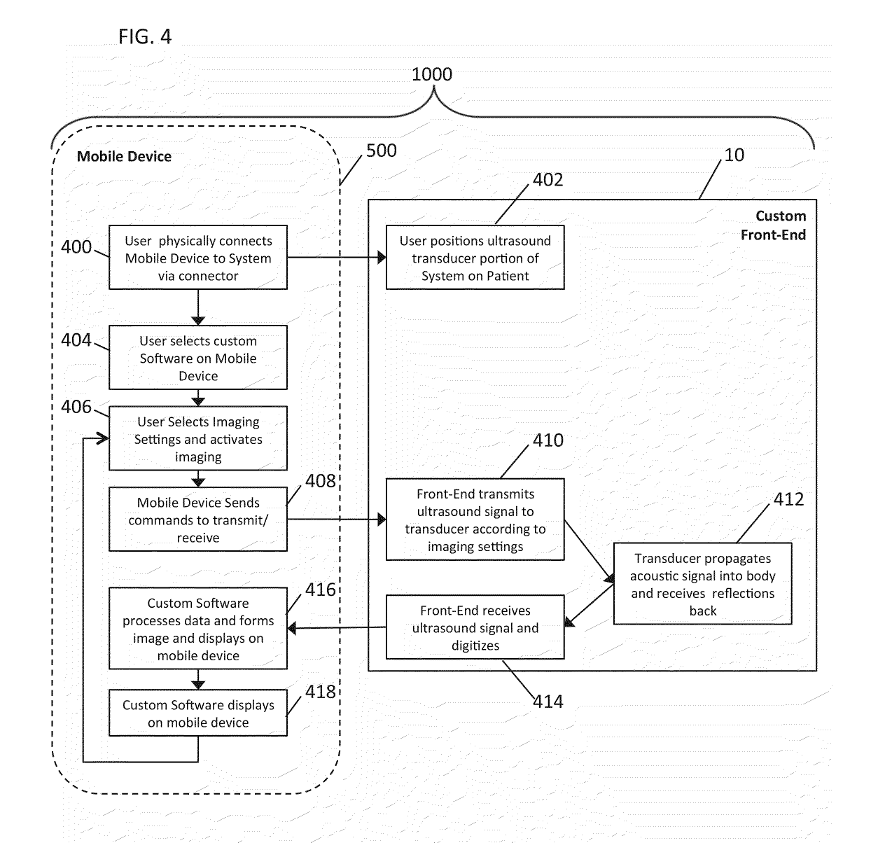

FIG. 4 is a flow chart illustrating events that may occur during operation of the system 1000 to provide ultrasonic imaging of a target location within a patient, according to an embodiment of the present invention. The order in which the events are described are not necessarily limiting to the order in which events would be performed in an actual use of the system. For example, although positioning the transducer on a patient is described prior to selection of the custom software to the mobile device below, the user could alternatively select the custom software on the mobile device prior to positioning the transducer on the patient. Other examples of alternative sequences will also be apparent to one of ordinary skill in the art. At event 400, the user or operator of the system 1000 physically connects the front end device 10 to the mobile computing device 500, such as in a manner described above with regard to FIGS. 3A-3B. At event 402, the user positions the transducer array 18 on the patient, over an area generally believed to be the location of the target to be imaged. For example, if a particular blood vessel is to be imaged, the transducer array 18 is placed against the skin of the patient overlying the location where the blood vessel to be imaged is believed to be located. Gel or other preparatory steps in placement of the transducer may be performed according to known, standard practices.

At event 404 the user selects the custom software on the mobile device to be used in performance of the imaging. Selection of the custom software may be performed, for example, by touching an icon 522 (see FIG. 3B) that represents the custom software to be activated and is configured to open the software upon touching the icon. Alternative means of selecting the software may be used, such as by operation of a keyboard or mouse, or various other equivalent alternatives that would be readily apparent to one of ordinary skill in the computer arts. Once the custom software opens, the user selects imaging setting and activates the software at event 406, using touch activation or other equivalent input controls. Upon activation, the mobile computing device 500, at event 408, sends commands through the communication interface 510 and communication hardware 12 to the transmit and receive control hardware 20, which control the custom ultrasound circuitry 16 to transmit ultrasound signals in accordance with the imaging settings to the transducer array 18 at event 410. At event 412, the transducer array 18 propagates acoustic signals into the body of the patient and received acoustic signals back from the body, the received signals having been reflected or echoed off of features within the patient's body.

At event 414, the custom circuitry receives electrical signals having been converted by the transducer array 18 from the received acoustic signals, and digitizes the electrical signals. At event 416, the data capture software 32 processes the digitized signals and forms image data that is sent via the data capture hardware 14 and communication hardware 12 to the mobile computing device, where the custom software 40 and CPU 502 cooperate to display an ultrasound image resulting from the processing on display 516 at event 418.

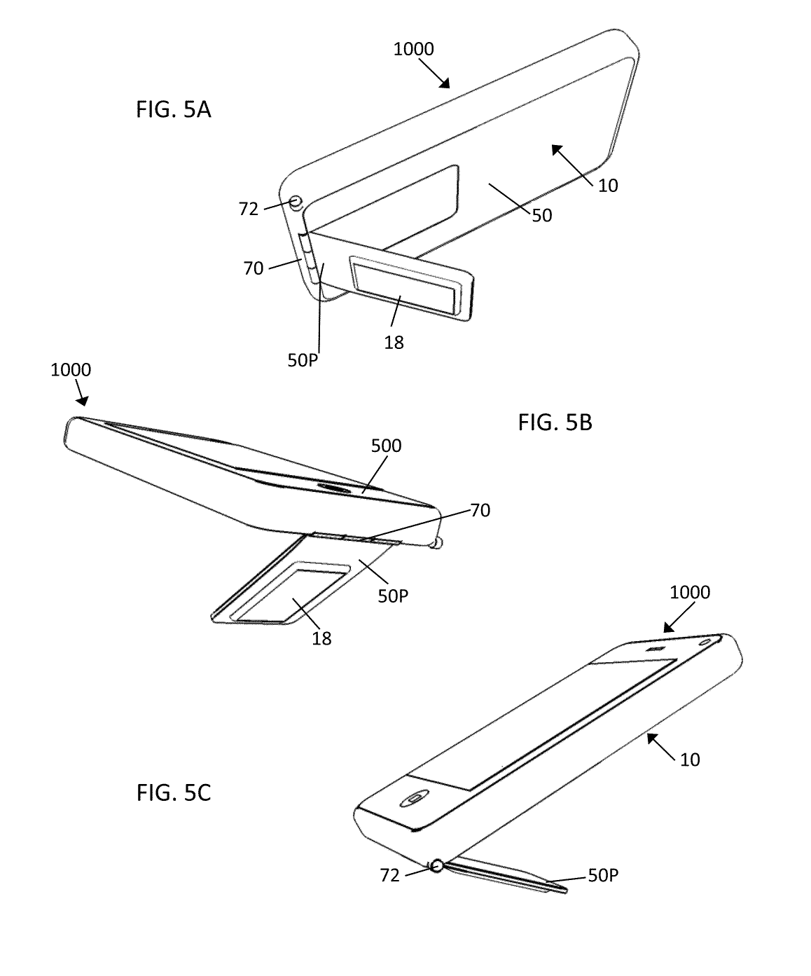

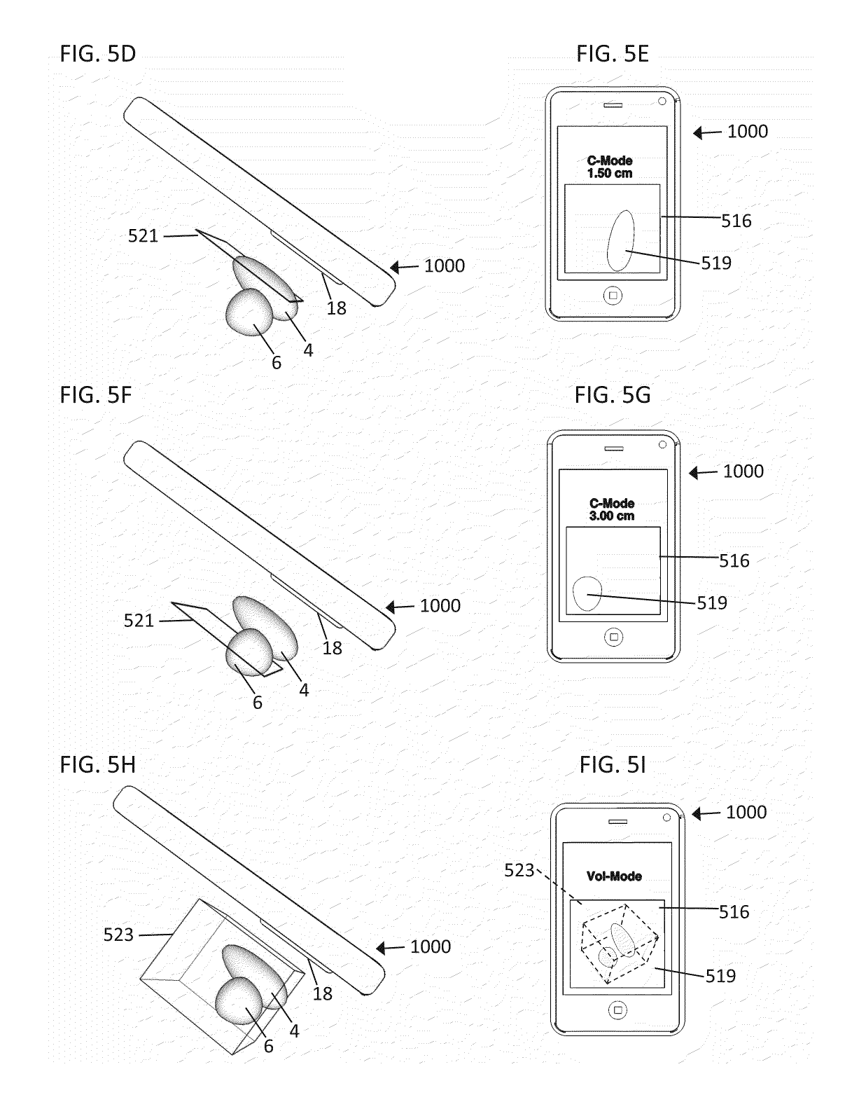

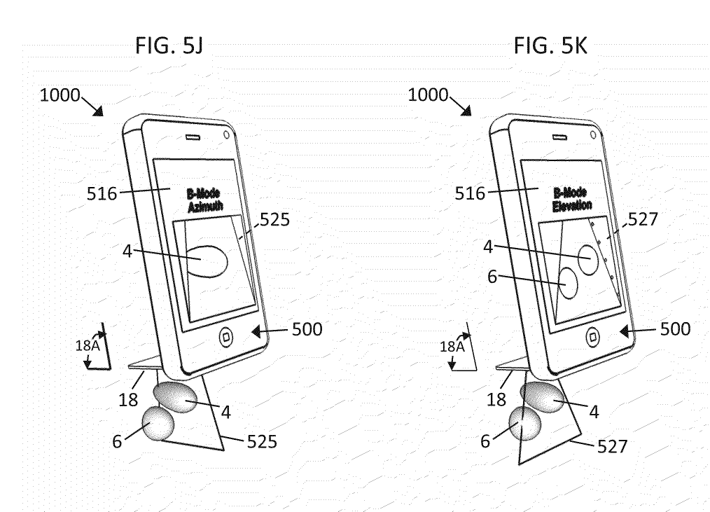

FIGS. 5A-5C are three different perspective views of a system 1000 illustrating angular adjustability of the transducer 18 relative to the mobile computing device 500 and front and back walls of the front end device 10, according to an embodiment of the present invention. In this and all embodiments described herein, the transducer 18 can be a single transducer element or consist of a plurality of transducer elements arranged in a one-dimensional array or two-dimensional array. The choice of number and arrangement of transducer elements will depend on such factors as the desired field of view, desired range and need for volume data required by the particular clinical application. FIG. 5A illustrates the transducer 18 in a position in which the transducer 18 is angled relative to the main walls of the front end device 10 and the front and back surfaces of the mobile computing device 500. It is noted that this is for illustration purposes only, as the transducer 18 can assume any angle between zero degrees (parallel to the main walls of the device 500 and main walls of the front end device 10 and 180 degrees relative to these walls. In other embodiments, the maximum angulation may be 170 degrees, 160 degrees, 90 degrees, or any value between 90 and 180 degrees. The range of relative transducer angles offered by the device will depend on the clinical application the device is designed to be used for. A portion 50P of the front end device 10 that the transducer 18 is mounted on or in is pivotally mounted to the remainder of the front end device 10 by hinge 70 in the embodiment of FIGS. 5A-5C. This arrangement enables the transducer to be positioned in an orientation at any angle to the wall 50 and front and back surfaces of the mobile computing device 500, preferably within the range of about 0 degrees to about 180 degrees, as noted above. At zero degrees, the transducer array is positioned flat, parallel to the front and back surfaces of the mobile computing device 500. In FIGS. 5A-5C, the transducer array 18 is shown at an angle of about forty degrees relative to the front and back faces of the mobile computing device 500. The hinge 70 may be provided with sufficient friction so that it allows changing the angle of transducer array 18 relative to the mobile computing device 500 by hand, but once positioned, the angle of the transducer array 18 is maintained until the user once again repositions it. This would function much like the hinge on a laptop computer, where the user can readily manually set the angular position of the display of the laptop computer relative to the keyboard, and this angular position maintains itself until the user chooses to reposition or close the laptop. Alternatively or additionally, a locking mechanism 72 such as a set screw, wing nut, or other mechanism can be provided which can be actuated by the user to further increase the friction in the hinge and securely lock the angular orientation of the transducer array 18 relative to the mobile computing device 500. In this arrangement, even if the array is accidentally bumped against something else or too much pressure is used in applying it to the patient, the angular orientation will be maintained if the locking mechanism has been actuated and locked. Upon releasing or unlocking the locking mechanism, the array 18 can again be repositioned.

The custom device software programming 30 is configured so that, when a position/orientation of the transducer 18 relative to the mobile computing device is changed, the system 1000 executes the software programming to change a display mode of an image being displayed. For example, FIGS. 5D-5I show the transducer array 18 oriented at an angle of zero degrees (i.e., aligned with) relative to the mobile computing device 500. In this orientation, C-Mode imaging is displayed at FIGS. 5D-5G, according to an embodiment of the present invention. Two generic three dimensional objects 4 and 6 are schematically illustrated in FIGS. 5D-5I as targets to be imaged. The first target 4 is longer and closer to the transducer 18 and the second target 6 is smaller and further from the transducer 18. Each of FIGS. 5D, 5F, 5H shows the position of these targets relative to the bottom of the system 1000/device 500/front end 10 and transducer 18 with a different imaging area and the corresponding display 516 in FIGS. 5E, 5G, 5I (relative to FIGS. 5D, 5F and 5H, respectively). In FIG. 5E, a two-dimensional image 519 of the target 4 is shown on the display 516 of the mobile computing device 500/system 1000 as a C-Mode image formed at a depth of 1.5 cm (into the patient, measured from the skin of the patient against which the transducer 18 is applied). The image plane at 1.5 cm depth is illustrated as 521 in FIG. 5D. At this depth, only the nearer/higher target 4 is visualized in the image plane 521. In FIG. 5G, a two-dimensional image 519 of the target area is shown on the display 516 of the mobile computing device as a C-Mode image taken at a depth of 3.00 cm. At this lower depth only the smaller target 6 is visualized in the image plane (see image plane 521 in FIG. 5F) and the higher target 4 is not seen. In FIG. 5I, a volume rendering image 519 of the two targets 4 and 6 is shown on the display 516 of the mobile computing device 500/system 1000 as a real-time volume image (image cube 523) is formed. A real time volume image is formed and displayed by combining multiple C-mode images taken at different depths/image planes 521 and integrated/combined.

FIGS. 5J and 5K show the transducer array 18 oriented at an angle 18A of ninety degrees relative to the mobile computing device 500 according to an embodiment of the present invention. In this orientation B-Mode imaging is displayed, according to an embodiment of the present invention. In another embodiment of this invention where a 2D array is used, it would be possible to display two separate B-mode images which are orthogonal planes corresponding to the Azimuthal (long-axis) B-Mode imaging plane (FIG. 5J) or the Elevational (short-axis) B-Mode imaging plane (FIG. 5K), where the plane 525 shown in FIG. 5J is orthogonal to the plane 527 shown in FIG. 5J. The targets 4 and 6 are schematically illustrated in FIGS. 5J-5K below the transducer 18. In FIG. 5J, a transverse, cross-sectional image is shown on the display 516 of the mobile computing device as a B-Mode Azimuth image. In FIG. 5J, the azimuth imaging plane 525 visualizes the cross section of the longer target 4 but does not visualize the other target 6. In FIG. 5K, a two-dimensional image 519 of the targets 4, 6 is shown on the display 516 of the mobile computing device as a B-Mode Elevation image. In FIG. 5K, the elevation imaging plane 527 visualizes the orthogonal cross section of both targets 4, 6 and the two dimensional image 519 shows that the target 6 is lower than the target 4. Switching between the B-Mode Azimuth image and the B-Mode Elevation image can be accomplished through the application software user interface such as by touching, pressing or otherwise actuating a soft key button, for example on the display 516 or other interface.

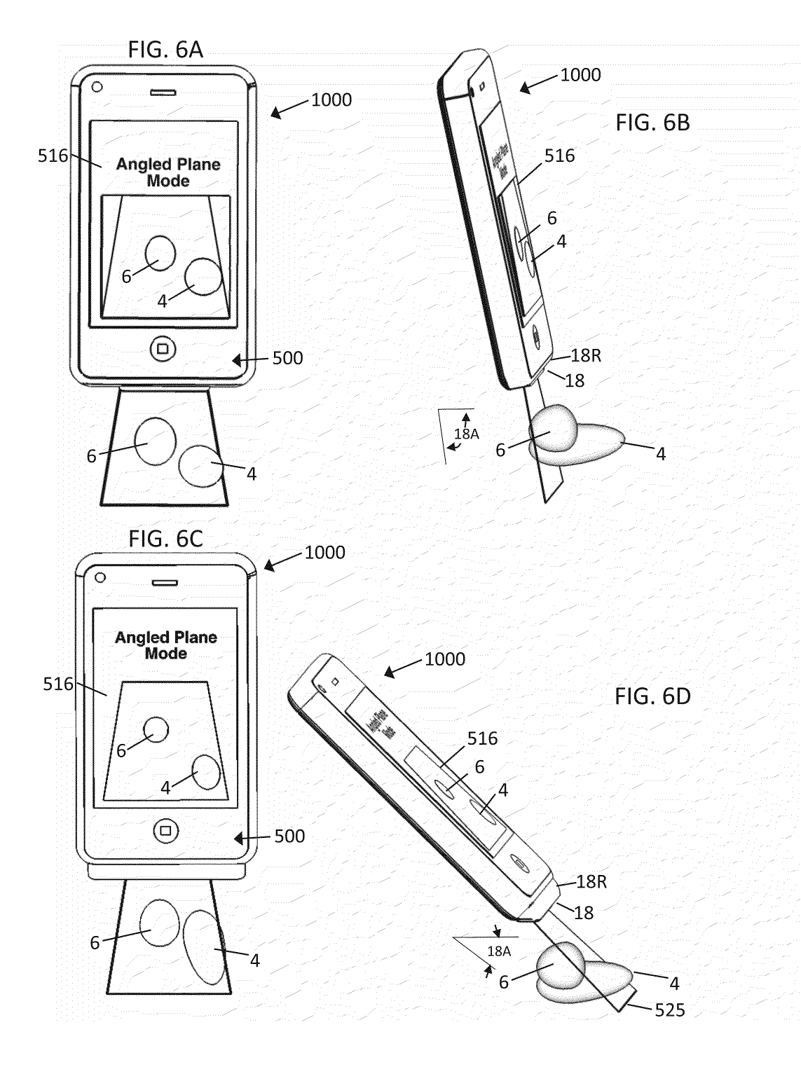

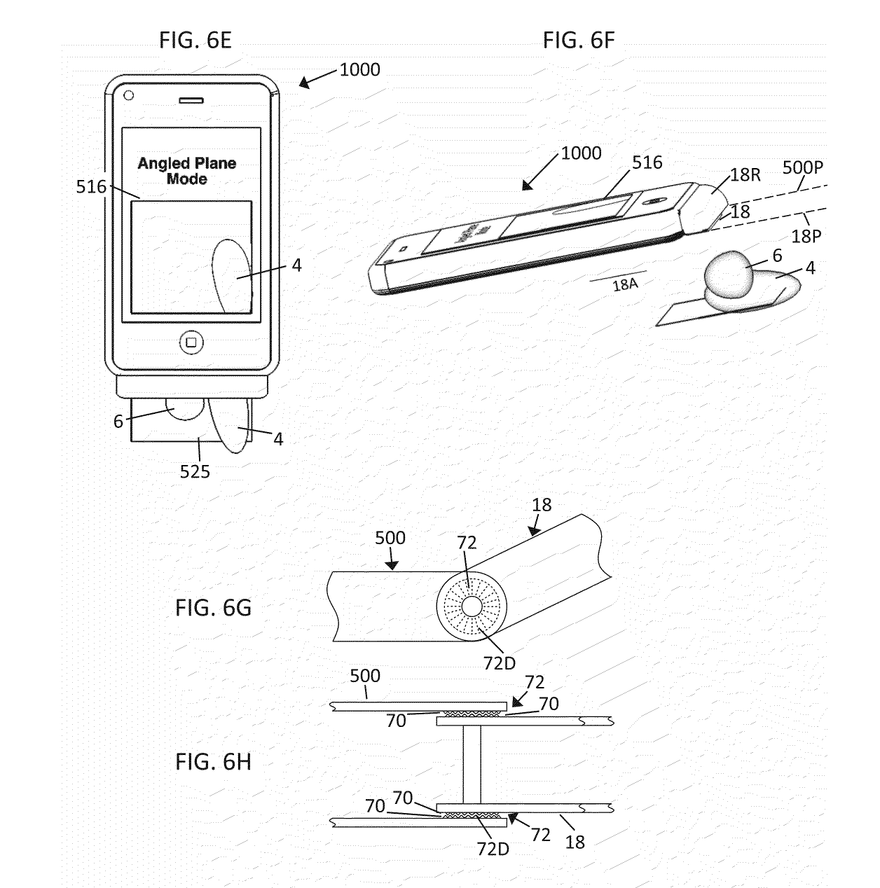

FIGS. 6A-6F illustrate respective front and side views of a system 1000 according to another embodiment of the present invention, in three different exemplary, but non-limiting use orientations. In this embodiment, the front end device 10 is configured such that the transducer array 18 is mounted at the end of the mobile computing device 500, as contrasted with mounting the transducer assembly 18 on the back side of the mobile computing device 500 as in the embodiment of FIGS. 5A-5K. Accordingly, when the transducer 18 is flush with the end of the mobile computing device 500, as shown in FIG. 6A (front) and FIG. 6B (side), it is oriented at an angle 18A of ninety degrees relative to the face of the display 516 and front and back surfaces of the mobile computing device, and B-Mode imaging is performed. In FIGS. 6A-6F, imaging of two generic targets 4 and 6 is shown, as in FIGS. 5A-5K, however the positioning of the two targets is not the same. In FIGS. 6A-6F, the longer target 4 is lower than the smaller target 6. In the transducer orientation shown in FIGS. 6A-6B, a two-dimensional image of the two generic targets 4 and 6 is shown on the display 516 of the mobile computing device as a B-Mode Azimuth image (seen in FIG. 6A), as the transducer array 18 is oriented at a ninety-degree angle to the surface of the display 516. The transducer array 18 is mounted on a rotating base 18R that rotates relative to the remainder of the front end device 10 to permit angling of the transducer array 18 relative to the mobile computing device. The rotating base 18R may be a hinged device, flexible membrane, accordioned structure, or other structure that permits angulation of the transducer array 18 relative to the display 51 at angles between and including zero and ninety degrees (while the longitudinal axis of the transducer array 18 remains normal to the longitudinal axis of the mobile computing device], and is configured to maintain the transducer array 18 at any of these angles until such time as the user chooses to reorient the transducer array 18 at a different angle. Like the previous embodiment, a locking mechanism may be provided to further assure that the intended angulation of the transducer assembly 18 relative to the display 516 does not change once it is selected (e.g., see FIGS. 6G-6H).

For transducer and mobile device orientations which are between the 0 and 90 degree orientations, in one embodiment of this invention this software program would be configured to detect the angled orientation and display an imaging plane corresponding to that particular orientation of the display plane relative to the transducer array face plane. As the orientation angle 18A of the transducer with the mobile device is changed a different imaging plane would be displayed. This angled plane imaging mode is different from the standard C-mode and B-mode imaging planes previously described and accepted by those skilled in the art. FIGS. 6C-6D show an example of this with the transducer array 18 oriented at an angle 18A of approximately 45 degrees relative to the display 516 (see FIG. 6D). This results in an angled plane mode image of both targets 4 and 6 being displayed on the display 516 (see FIG. 6C).

FIGS. 6E-6F show the transducer array 18 at an angle 18A of about zero degrees relative to the display face 516, as the plane 18P of the face of the array 18 and the plane 500P of the display face 516 are substantially parallel, as illustrated in FIG. 6F. As a result, a C Mode image is displayed and when the image is formed at a depth of about 3.0 cm as in FIGS. 6E-6F, only the larger and lower target 4 is visualized in the image (see FIG. 6E).

FIGS. 6G-6H illustrate an example of a locking element according to an embodiment of the present invention. In this embodiment, locking element 72 is an internal mechanism configured to fix the array 18 at a desired angular position relative to the display 516. FIG. 6G is a side view of the locking element 72 and FIG. 6H is a top view of the locking element 72. The locking element 72 comprises detents 72D on the surfaces of the hinge elements 70 which allow for movement when a force greater than a predetermined force is manually applied to the hinges (such as by manually moving the array 18 while holding the device 500 relatively stationary), but which maintains the array 18 fixed relative to the display 516 at the angle manually set during an imaging procedure. The entire hinge 70 and locking element 72 will be covered with a flexible membrane to maintain cleanliness as is shown in FIG. 6A-6F, for example.

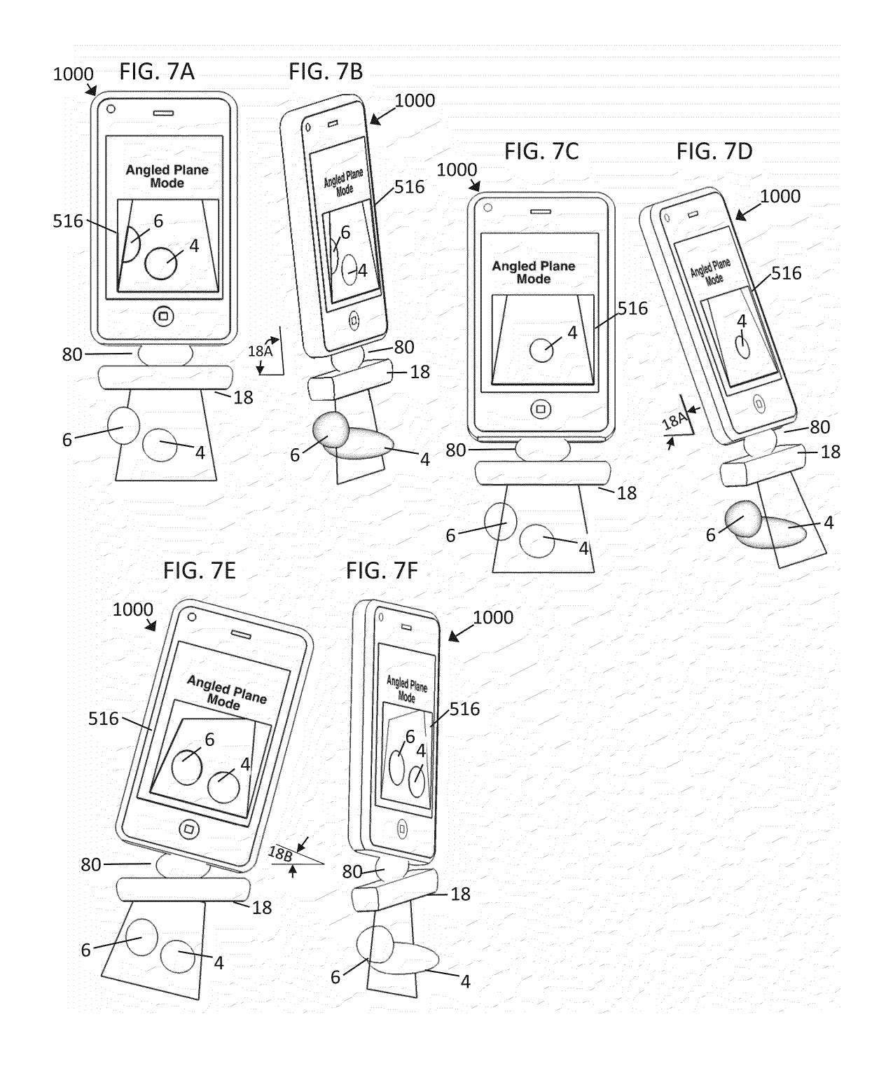

FIGS. 7A-7F illustrate a system 1000 according to another embodiment of the present invention, in which the front end device 10 includes a transducer assembly 18 that is mounted on the end of the mobile computing device 500, like in the embodiment of FIGS. 6A-6F. However, in this embodiment, transducer assembly 18 is mounted on joint 80 that enables articulation of the transducer array 18 relative to the display 516 in all three dimensions. Preferably joint 80 is a ball joint assembly, although other alternative joints providing three degrees of freedom could alternatively be employed. Preferably the joint 80 allows repositioning of the orientation of the array 18 relative to the device 500, but has sufficient friction to maintain the array 18 in the orientation that it is manually placed in until the user manually repositions/reorients the array 18. In each of the FIGS. 7A-7F, the same two targets 4 and 6 are shown below the transducer array 18, as shown in FIGS. 6A-6F, and the system 1000 is repositioned in different orientations. In FIGS. 7A-7B the face of the transducer array 18 is normal to the plane of the display 516 (i.e., angle 18A of about 90 degrees) and the device display 516 is showing a B-mode image corresponding to the same plane as the display (similar to mode shown in FIGS. 6A-6B). In this configuration the imaging plane visualizes a cross section of target 4 and a portion of target 6. In FIGS. 7C-7D, the device is angled backwards relative to the transducer array 18 (approximately 45 degrees, angle 18A, FIG. 7D), similar to that described in regard to FIGS. 6C-6D above, and the angled image plane is displayed. Due to the change in angle of the device, the imaging plane, which is in the same plane as the display of the device, now visualizes only the front portion of the longer target 4 and no portion of the shorter target 6. In FIGS. 7E-7F, the device is rotated approximately 15 degrees laterally relative to the transducer array 18 as illustrated by angle 18B in FIG. 7E. In this orientation, the plane of the face of the array 18 is about 90 degrees relative to the plane of the face of the array 18 (as in 7A-7B), but the transverse axis of the face of the display 516 is angled by angle 18B relative to the plane of the face of the array 18. In this embodiment, the rotation of the device again steers the image plane at the same angle as the angle of the plane of the display and the resultant imaging plane is displayed on the device, as illustrated in FIGS. 7E-7F. Because of this angled plane the displayed image now captures the entire cross section of the smaller target 6 as well as the target 4, with both targets moving towards the right of the image (as seen in comparing FIG. 7E to FIG. 7A).

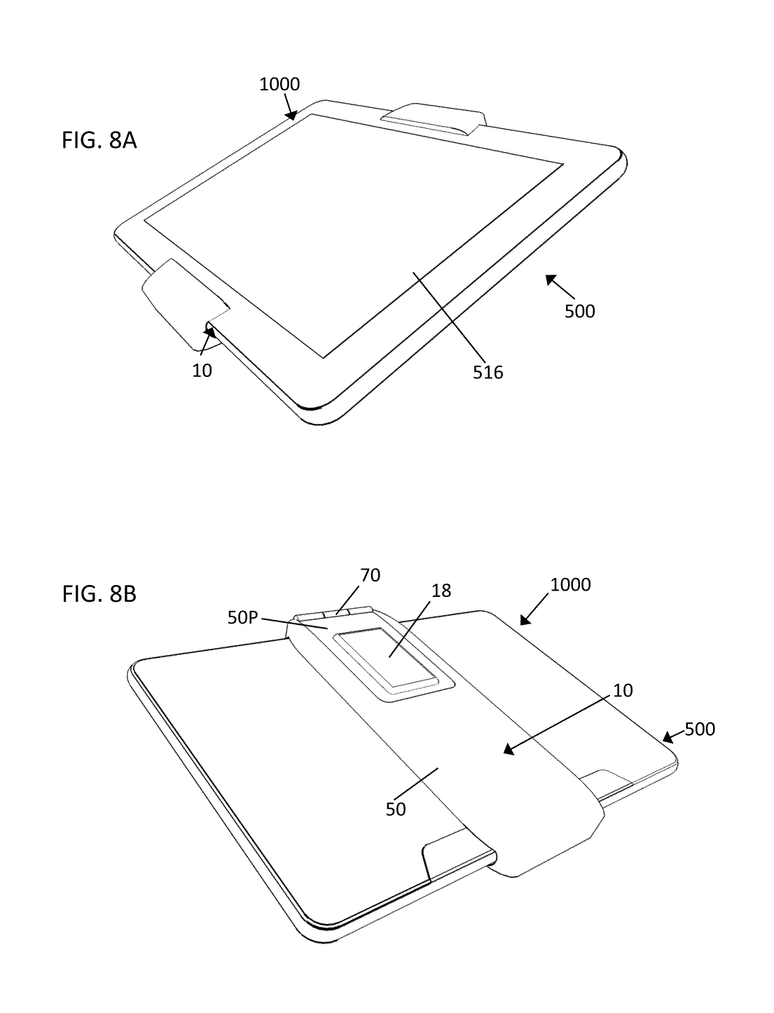

FIGS. 8A-8B illustrate a system 1000 according to an embodiment of the present invention wherein the mobile computing device 500 comprises a tablet computer, such as an IPAD or the like. In FIG. 6B. This embodiment functions in the same manner as the embodiment shown in FIG. 5A, with the difference being that the front end device 10 of this embodiment does not surround the entire mobile computing device, but instead spans it on only two ends (e.g. from top to bottom). Connection of the front end device 10 to the mobile computing device 500 is essentially the same in this embodiment as in the embodiment of FIG. 5A, except that the device 10 seals against the back surface of the mobile computing device 500, rather than along its side edges.

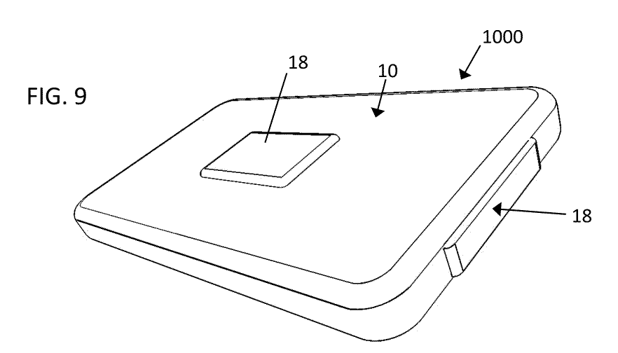

FIG. 9 illustrates an embodiment in which the front end device is provided with two transducer arrays 18, one at the end and one on the back surface of the front end device 10. Two arrays allow for different orientations, without the need to rotate a single array, or allow for different frequencies on the same device. The two transducers 18 could be various one and two dimensional arrays and also be oriented on different faces of the device. The exact configuration would depend on the clinical applications the device would be used for, but as an example two different transducer frequencies would allow for the single device to image a wider range of clinical applications (as a parallel to a conventional cabled device which allows for transducers to be swapped out depending on the application). In the case of two transducers with distinct center frequencies, a lower frequency transducer on the back (e.g. 4 MHz center frequency) would allow for greater penetration when the clinical application or patient type requires it. A higher frequency transducer (e.g. 7 MHz center frequency) on the end would allow for more detailed (higher resolution) imaging when shallower imaging is required. It is noted here that the frequencies mentioned above are only an example and may vary according to the particular procedures to be performed.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A portable ultrasound imaging system comprising:
a mobile computing device;
a detachable front end component configured for attachment to and communication with said mobile computing device, and configured to transmit and receive ultrasound signals; and
programming, when installed on said mobile computing device, being executable by said mobile computing device to cause said mobile computing device to send signals to said front end component causing said front end component to transmit said ultrasound signals, and to receive signals from said front end component resulting from said front end component receiving said receive ultrasound signals, and process said receive signal and display an ultrasound image resulting from said processing;
wherein said front end component is configured to be directly joined with said mobile computing device and directly connected, without the use of an external wire or cable.

2. The system of claim 1, wherein at least a portion of said front end component is movably mounted to said mobile computing device to allow relative rotation about at least one axis of rotation relative to said mobile computing device.

3. The system of claim 1, wherein said mobile computing device is a device selected from the group consisting of: a smartphone, a tablet computing device, and a personal digital assistant (PDA).

4. The system of claim 1, wherein said mobile computing device comprises a smartphone.

5. The system of claim 1, wherein said mobile computing device comprises a tablet computing device.

6. The system of claim 1, wherein said at least a portion of said front end component is movably mounted to said mobile computing device to allow relative rotation about at least two axes of rotation relative to said mobile computing device.

7. The system of claim 1, wherein at least a portion of said front end component is movably mounted to said mobile computing device to allow relative rotation about three axes of rotation relative to said mobile computing device.

8. The system of claim 1, wherein said ultrasound image is displayed in real-time.

9. The system of claim 1, wherein said front end component further comprises a barrier element that shields said mobile computing device from contact with a patient when said front end component is applied to a patient.

10. The system of claim 9, wherein said barrier element forms a seal with said mobile computing device to provide a sterile bather.

11. The system of claim 2, further comprising a locking element configured to fix said front end component relative to said mobile computing device to maintain a desired orientation of said front end component relative to said mobile computing device.

12. The system of claim 2, wherein said programming is configured so that, when a position of front end component relative to said mobile computing device is changed, said processor executes said programming to change a display mode of an image being displayed.

13. The system of claim 1, wherein said front end component comprises a two-dimensional ultrasound transducer.

14. The system of claim 13, wherein execution of said programming by said processor processes said receive signals to form an image similar to an image that would otherwise be formed by processing signals received from a front end component employing a one-dimensional transducer.

15. The system of claim 1, wherein said front end component comprises a one-dimensional ultrasound transducer.

16. The system of claim 1, wherein said front end component comprises multiple distinct transducer arrays which are capable of acquiring two separate sets of ultrasound data, each said distinct transducer array being configured to transmit and receive distinct ultrasound signals.

17. The system of claim 16, wherein a first of said two distinct transducer arrays operates at a first center frequency, and a second of said two distinct transducer arrays operates at a second center frequency, wherein said second center frequency is different from said first center frequency.

18. The system of claim 16, wherein a first of said two distinct transducer arrays is a one-dimensional transducer array, and a second of said two distinct transducer arrays is a two-dimensional transducer array.

19. The system of claim 16, wherein said two distinct transducer arrays are oriented in different directions on said front end component.

20. A front end component configured for communication with a mobile computing device to function as a portable ultrasound imaging system, said front end component comprising:
a main body configured and dimensioned to fit over the mobile computing device;
a mating connector configured and dimensioned to directly mate with a connector on the mobile computing device for direct connection of the front end component to the mobile computing device without any need for a connection wire or cable; and
a transducer array movably mounted relative to said main body, to allow relative rotation of said transducer array about at least one axis of rotation relative to said main body;
wherein said main body is configured to form a seal with the mobile computing device.

21. The front end component of claim 20, wherein said transducer array is configured for a predetermined footprint and element pitch; and wherein said front end comprises at least one application specific integrated circuit (ASIC) configured to enable a front end dimensional footprint and front end channel pitch, wherein said front end dimensional footprint and said front end channel pitch match said footprint and element pitch, respectively.

22. A method of operating a portable ultrasound imaging system comprising:
directly connecting a front end device to a mobile computing device, without the use of an extension cable or wire, the front end component including a transducer array, configured for communication with the mobile computing device, and configured to transmit and receive ultrasound signals;
positioning the transducer array over a location of a target to be imaged;
selecting custom software installed on the mobile computing device to be used in performance of imaging;
selecting imaging settings on the custom software;
activating the custom software;
propagating acoustic signals toward the target to be imaged;
receiving acoustic signals reflected off of the target to be imaged;
converting the acoustic signals received to digitized electrical signals;
processing the digitized electrical signals; and
displaying an image of the target on a display of the mobile computing device.

23. The method of claim 22, wherein the transducer, in a first angular orientation relative to the display, causes the custom software to display in a first imaging mode.

24. The method of claim 23, further comprising changing the transducer to a second angular orientation relative to the display, wherein the second angular orientation causes the custom software to display the image in a second imaging mode different from said first imaging mode.

25. A non-transient computer readable medium including one or more sequences of instructions for performing ultrasound imaging system on a portable ultrasound imaging system, wherein execution of the one or more sequences of instructions by one or more processors of the portable ultrasound imaging system causes the portable ultrasound imaging system to perform a process comprising:
setting imaging settings for an imaging process to be performed on a mobile computing device loaded with said one or more sequences of instructions, and directly connected to a front end device including a transducer array;
sending commands from the mobile computing device to a transmit and receive control module in the front end device;
controlling ultrasound circuitry to transmit ultrasound signals in accordance with the imaging settings to the transducer array;
propagating acoustic signals into a target to be imaged;
receiving acoustic signals having been reflected off the target to be imaged;
converting the acoustic signals received to electrical signals;
processing the electrical signals; and
displaying an image of the target on a display of the mobile computing device.

26. The non-transient computer readable medium of claim 25, further including instructions which, when executed by the portable ultrasound imaging system, cause the system to:
display the image in a first imaging mode when the transducer is in a first angular orientation relative to the display; and
upon changing orientation of the transducer array relative to the display to a second angular orientation different from said first angular orientation, displaying the image in a second imaging mode different from said first imaging mode.
